(19) United States
(12) Patent Application Publication
(10) Pub. No.: US 2021/0137681 A1
(43) Pub. Date: May 13, 2021

(54) Delivery Systems and Methods fro Transseptal Access to a Left Atrium

(71) Applicant: Medtronic Vascular, Inc., Santa Rosa, CA (US)

(72) Inventors: Michael BATEMAN, Santa Rosa, CA (US); George MALLIN, Santa Rosa, CA (US); James R. KEOGH, Santa Rosa, CA (US)

(21) Appl. No.: 16/486,417

(22) PCT Filed: Mar. 16, 2018
(86) PCT No.: PCT/US2018/022786

§ 371 Date: Aug. 15, 2019

Related U.S. Application Data
(60) Provisional application No. 62/473,705, filed on Mar. 20, 2017.

(51) Int. Cl. A61F 2/24; A61B 18/14

(52) U.S. Cl. CPC A61F 2/2433; A61B 2018/00577; A61B 18/1492; A61F 2/2436

(57) ABSTRACT

Delivery systems (100, 200, 300, 400, 500) including a delivery device (130, 230, 330, 430, 530) having a catheter (132, 133, 232, 233, 332, 333, 432, 433, 532) and a stabilization device (150, 250, 350, 450, 550), the delivery device configured for delivering an apparatus to a left atrium (20) of a patient via the patient's vasculature. The stabilization device has a delivery position in which the stabilization device is collapsed against the catheter and a deployed position in which the stabilization device expands to engage and support the catheter within the vasculature, for example, within the vena cava (14) proximate the right atrium (16). In various embodiments, the delivery device functions as an introducer though which a second delivery device carrying the apparatus is inserted. Methods of accessing the left atrium with the delivery systems and devices are also disclosed.

### CROSS-REFERENCE TO RELATED APPLICATIONS

This Patent Application claims the benefit of the filing date of U.S. Provisional Patent Application Ser. No. 62/473,705, filed Mar. 20, 2017, entitled “DELIVERY SYSTEMS AND METHODS FOR TRANSSEPTAL ACCESS TO A LEFT ATRIUM,” the entire teachings of which are incorporated herein by reference.

### BACKGROUND

The present disclosure relates to systems and methods for accessing a left atrium of a human heart. More particularly, it relates to systems and methods for transseptally accessing a left atrium of a heart for delivery of an apparatus, such as a prosthesis, ablation apparatus or other apparatus.

The heart is a four-chambered pump that moves blood efficiently through the vascular system. Blood enters the heart through the vena cava and flows into the right atrium. From the right atrium, blood flows through the tricuspid valve and into the right ventricle, which then contracts and forces blood through the pulmonic valve and into the lungs. Oxygenated blood returns from the lungs and enters the heart through the left atrium and passes through the mitral valve into the left ventricle. The left ventricle contracts and pumps blood through the aortic valve into the aorta and to the vascular system.

Diseased or otherwise deficient heart valves can be repaired or replaced with an implanted prosthetic heart valve. Conventionally, heart valve replacement surgery is an open-heart procedure conducted under general anesthesia, during which the heart is stopped and blood flow is controlled by a heart-lung bypass machine. Traditional open-heart surgery inflicts significant patient trauma and discomfort, and exposes the patient to a number of potential risks, such as infection, stroke, renal failure, and adverse effects associated with the use of the heart-lung bypass machine, for example.

Due to the drawbacks of open-heart surgical procedures, there has been an increased interest in minimally invasive and percutaneous replacement of cardiac valves. With these percutaneous transcatheter (or transluminal) techniques, a valve prosthesis is compacted for delivery in a catheter and then advanced, for example, through an opening in the femoral artery and through the patient's vasculature to the target site. A common approach for accessing the left side of the heart is a transseptal access from the right atrium through the intra-atrial septum to the left atrium.

The disclosure addresses problems and limitations associated with related delivery systems and devices for transseptally accessing a left atrium.

### SUMMARY

One aspect of the present disclosure relates to a delivery system including a delivery device having a catheter and a stabilization device. An apparatus for treating a human heart can be secured to a distal end of the catheter. The stabilization device has a delivery position in which the stabilization device is collapsed against the catheter and a deployed position in which a diameter of the stabilization device is expanded. In the expanded deployed position, the stabilization device supports and secures the catheter in position so that the catheter does not apply potentially detrimental pressure or friction to the patient's anatomy, for example, at the inferior vena cava or right atrium. The disclosed stabilization devices can take a number of configurations that provide the delivery and deployed positions. For example, the stabilization device can comprise an inflatable balloon or can comprise ribs that can be actuated to bow outwardly with respect to the catheter.

Another aspect of the present disclosure relates to a method of delivering an apparatus, such as a prosthesis, ablation or other apparatus, to a left atrium via transseptal delivery. The method includes providing a delivery system including a delivery device having a catheter and a stabilization device. As the delivery device is directed to a patient's right atrium, the stabilization device is placed in the delivery position, collapsed against the catheter. Once in position, the stabilization device is actuated to transition from the delivery position to an expanded, deployed position to engage and support the catheter. In various embodiments, the stabilization device is deployed so that the stabilization device engages the patient anatomy proximate the intersection of the inferior vena cava and the right atrium.

In alternate embodiments, a delivery system includes a first delivery device including a stabilization device secured to a catheter that is configured as a hollow tube. The first delivery device functions as a stabilized introducer catheter for guiding a second delivery device carrying the prosthesis, ablation apparatus or other apparatus. Once the first delivery device is transseptally delivered to the left atrium and stabilized within the vena cava with the deployed stabilizing device, the second delivery device carrying the apparatus is inserted through the catheter and directed to the left atrium. Once a treatment procedure within the left atrium is complete, the stabilization device can be transitioned to the delivery position and then the first and second delivery devices are withdrawn.

### BRIEF DESCRIPTION OF THE DRAWINGS

FIG. 1 is a schematic illustration of a procedure for transseptally delivering an apparatus to a patient's left atrium via the patient's vena cava and right atrium.

FIG. 2A is a partial, schematic illustration of a delivery system including a delivery device carrying a prosthesis, the delivery device having catheters and a stabilization device; wherein the stabilization device is in a delivery position.

FIG. 2B is a partial, schematic illustration of the delivery system of FIG. 2A illustrating the stabilization device of the delivery device in an expanded, deployed position.

FIG. 2C is a cross-sectional schematic illustration of the delivery device of FIG. 2B as viewed along line 2C-2C; wherein the stabilization device is deployed within the vena cava (shown in phantom).

FIG. 3A is a partial, schematic illustration of an alternate delivery system including a delivery device carrying a prosthesis, the delivery device having catheters and a stabilization device; wherein the stabilization device is in a delivery position.

FIG. 3B is a partial, schematic illustration of the delivery system of FIG. 3A illustrating the stabilization device of the delivery device in an expanded, deployed position.

FIG. 3C is a cross-sectional schematic illustration of the delivery device of FIG. 3B as viewed along line 3C-3C; wherein the stabilization device is deployed within the vena cava (shown in phantom).

FIG. 4A is a partial, schematic illustration of an alternate delivery system including a delivery device carrying an ablation apparatus, the delivery device having a catheter and a stabilization device; wherein the stabilization device is in a delivery position.

FIG. 4B is a partial, schematic illustration of the delivery device of FIG. 4A illustrating the stabilization device in an expanded, deployed position.

FIG. 4C is a cross-sectional schematic illustration of the delivery device of FIG. 4B as viewed along line 4C-4C with the stabilization device deployed within the vena cava (shown in phantom).

FIG. 5 is a schematic illustration of a delivery device of an alternate delivery system including a delivery device delivering a prosthesis to a left atrium, the delivery device including a catheter and a stabilization device; wherein the stabilization device is in an expanded, deployed position.

FIG. 6 is a cross-sectional schematic illustration of the delivery device of FIG. 5 illustrating the deployed stabilization device of FIG. 5 as viewed along line 6-6.

FIG. 7 is a schematic illustration of an alternate delivery system including a delivery device having a catheter and a stabilization device, such as any of the disclosed stabilization devices, wherein the catheter can be hollow to serve as an introducer for guiding a second delivery device of the delivery system to a left atrium.

### DETAILED DESCRIPTION

Specific embodiments of the present disclosure are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

By way of background, one example of a procedure for treating a patient 10 having a human heart 12 including a vena cava 14, right atrium 16, septal wall 18, left atrium 20 and a plurality of valves, including a mitral valve 22, is generally depicted in FIG. 1. In this example, a delivery device 30 including one or more coaxially arranged catheters 32 supporting an apparatus (not visible) is maneuvered, perhaps routed through the vena cava 14 to the right atrium 16 and to the patient's septal wall 18. As illustrated, the delivery path can continue transseptally through the patient's septal wall 18 to the left atrium 20 and resume by turning approximately 90° downward through the native mitral valve 22. The catheter 32 is tracked in over a guide wire 36 in a direction indicated by arrow Ai to position a distal end and tip 34 of the delivery device 30 (including the apparatus) through and past the native valve 22. One of the difficulties in either positioning the prosthesis or other apparatus in the left atrium 20 or crossing the mitral valve annulus 22 with a transcatheter delivery device is stabilizing the delivery device 30 or system during the procedure. Although the point at which the catheter 32 passes through the septal wall 18 acts in some way as a stabilization point for the catheter 32, heart tissue can be damaged during the articulation of the catheter tip 34 into the left atrium 20. Additionally, the catheter 32 almost inevitably pushes against the right side of the vena cava 14, just inferior to the right atrium 16 (assuming trans-femoral venous access), which can cause damage to the vena cava 14 and/or perhaps, other heart tissue. It is believed that the embodiments of the present disclosure will allow for atraumatic stabilization of a delivery device in the superior inferior vena cava causing less trauma to the inter atrial septum and the right atrium as the catheter tip is articulated in the left atrium. The disclosed embodiments are believed to improve catheter tip control for the physician and reduce damage to the inter-atrial septum and other tissue of the patient.

Turning now also to FIGS. 2A-2C, which illustrate select components of a delivery system 100 including a delivery device 130 having catheters 132 and 133 and a stabilization device 150 that can be used for transseptal access of a left atrium. As shown, the catheter 133 is coaxially aligned and slidable with respect to a catheter 132, which carries or otherwise supports an apparatus 140 at a distal end portion. In one embodiment, the delivery system 100 may include a sheath (not shown) coaxially aligned and slidable with respect to catheters 132 and 133. The sheath can reside within catheter 132 covers the catheter 133. The sheath may also include a distal capsule or portion for covering apparatus 140 during delivery. Alternatively, the sheath and catheter 133 may be omitted and catheter 132 can carry or otherwise support an apparatus 140 at a distal end thereof. The apparatus 140 can be a prosthesis, as illustrated, or can be an ablation apparatus or other apparatus suitable for treating a human heart. In one embodiment, the delivery device 130 may further include a sheath 134 that is coaxially aligned with and covers the catheter 132. In one embodiment, sheath 134 can cover catheter 132 with the exception of a region 136 over which the stabilization device 150 is positioned. The stabilization device 150 of this embodiment includes proximal and distal rings 152, 154 interconnected by a plurality of flexible ribs 156 (e.g., three ribs, equally spaced 120 degrees from one another about the rings 152, 154). The number of ribs 156 can differ, as desired. In one embodiment, the distal ring 152 is fixed to the catheter 132 and the proximal ring 154 is slidable with respect to the catheter 132.

FIG. 2A illustrates the stabilization device 150 in a delivery position in which the ribs 156 are straightened to collapse against the catheter 132 and provide a low profile for delivery through a patient's vasculature. Once the stabilization device 150 is in the desired position (e.g., proximate or at the inferior vena cava), the stabilization device 150 can be deployed into a deployed position so that it expands in diameter to engage the respective heart vessel wall and support the catheter 132 away from the respective vessel wall (e.g., wall of the inferior vena cava). In the deployed position shown in FIGS. 2B-2C, blood flow is not significantly obstructed by the stabilization device 150 as blood flow pathways 160 are formed between the ribs 156.

In this embodiment, the stabilization device 150 is deployed by pushing the proximal ring 154 toward the distal ring 152, which causes the ribs 156 to bow outwardly. The proximal ring 154 can be pushed, for example, by distally advancing the sheath 134 over the catheter 132. As the sheath 134 is distally advanced, the region 136 of the catheter 132 that is not covered by the sheath 134 is forced to shorten and subsequently causes the ribs 156 to bow outwards with respect to the catheter 132. The degree in which the ribs 156 bow outwardly is adjustable depending on how far the proximal ring 154 is pushed distally. Other ways of moving the proximal ring 154 toward the distal ring 152 to deploy the stabilization device 150 are envisioned. Once in the deployed position, the catheter 132 is substantially secured in position and is not easily advanced or withdrawn through the septal wall opening. After the apparatus 140 is implanted or other treatment procedure is complete, the sheath 134 can be proximally retracted, which will cause the ribs 156 to once again collapse against the catheter 132 to create a low profile for retraction of the delivery device 130.

In one embodiment, the distal end of the sheath 134 is coupled to the proximal ring 154 so that proximal and distal movement of the sheath 134 moves the proximal ring 154 proximally and distally. Distal movement of the sheath 134 pushes proximal ring 154 distally to expand the ribs 156 while proximal movement of sheath 134 allows the ribs 156 to straighten out and collapse to their biased configuration. Sheath 134 does not necessarily have to be coupled to the proximal ring 154 to function in this way.

In one or more embodiments, the sheath 134 may be omitted and substituted with one or more push/pull rods or wires (not shown) may be connected to the proximal ring 154 and/or the distal ring 152. The rods or wires may be used to deploy stabilization device 150 by either pushing the proximal ring 154 towards the distal ring 152, if the distal ring 152 is fixed to catheter 132 and the proximal ring 154 is slidable relative to catheter 132, or by pulling the distal ring 152 towards the proximal ring 154, if the proximal ring 154 is fixed to the catheter 132 and the distal ring 152 is slidable relative to catheter 132. In both embodiments, the ribs 156 may be biased to be in the straight delivery configuration and the degree in which the ribs 156 bow outwardly can be adjustable depending on how far the proximal ring 154 is pushed distally or the distal ring 152 is pulled proximally.

Turning now also to FIGS. 3A-3C, which illustrate select components of a delivery system 200 including a delivery device 230 having catheters 232 and 233 and a stabilization device 250 that can be used for transseptal access of a left atrium. In this embodiment, the stabilization device 250 includes proximal and distal rings 252, 254 interconnecting a plurality of ribs 256. The ribs 256 may be biased to be in the expanded deployed configuration. In this embodiment, a sheath 234 may cover catheter 232 as well as cover the stabilization device 250 in a delivery position. The sheath 234 can then be proximally retracted to uncover the stabilization device 250, thereby allowing the ribs 256 to expand or bow outwardly into a deployed configuration. The sheath 234 can then be advanced distally to cover the stabilization device 250 to once again collapse ribs 256 against the catheter 232 to create a low profile for retraction of the delivery device 230. In this embodiment, the proximal ring 254 may be fixed to the catheter 232 and the distal ring 252 may be slidable relative to the catheter 232.

Turning now also to FIGS. 4A-4C, which illustrate select components of another delivery system 300 that includes a delivery device 330 having catheters 332, 333 and an alternate stabilization device 350. In this embodiment, the catheter 333 is coaxially aligned and slidable with respect to the catheter 332. Catheter 333 carries an apparatus 340 at a distal end portion, such as an ablation apparatus, for delivery to a left atrium via a transseptal access procedure (e.g., the procedure as generally illustrated in FIG. 1). Alternatively, catheter 333 may be omitted and catheter 332 can carry or otherwise support an apparatus 340 at a distal end portion. In some embodiments, the stabilization device 350, as shown in FIG. 4A-4C, may be a balloon made of a nylon based polymer, polyurethane or the like that can be selectively inflated with any suitable gas or liquid, for example. FIG. 4A illustrates the stabilization device 350 in the delivery position, wherein the stabilization device 350 is deflated to collapse against the catheter 332 and does not substantially increase the profile of the catheter 332 for delivery. In the deployed position of FIGS. 4B-4C, the stabilization device 350 is inflated, revealing a generally triangular cross-section, which provides support for the catheter 332 while also providing for three blood flow pathways 360 when positioned within a generally tubular lumen of a vessel (e.g., the inferior vena cava 14, see, FIG. 4C). The stabilization device 350 can have other cross-sectional configurations that will provide support to the catheter 332 while still allowing blood flow past the stabilization device 350. Such alternatives are considered within the scope of the disclosure. The stabilization device 350 can be deflated prior to withdrawing the delivery device 330 from the patient after the procedure.

Referring now to FIG. 5, which illustrates select components of another delivery system 400 including a delivery device 430 having catheters 432 and 433 supported within the inferior vena cava 14 by an alternate stabilization device 450. It is noted that the stabilization devices 150, 350 disclosed above support the respective catheters 132, 332 in similar ways. FIGS. 5-6 illustrate that the stabilization device 450 of this embodiment includes a balloon 452 having a plurality of webs 454 interconnecting the balloon 452 to the catheter 432. In a delivery position (not shown), the balloon 452 is deflated and is collapsed against the catheter 432 to create a low profile for delivery. Once in position, the balloon 452 is inflated. In the deployed position shown in FIGS. 5-6, the balloon 452 is inflated to expand and define a generally cylindrical shape and the webs 454 further inflate and lengthen to push the balloon 452 away from the catheter 432. In the deployed position, three blood flow paths 460 are formed within the balloon 452 and between the webs 454, assuming that three webs 454 are provided. More or fewer webs 454 can be provided, as desired and the number of blood flow paths 460 will vary accordingly. As with the prior embodiment, the stabilization device 450 can be deflated after the procedure to disengage the stabilization device from the anatomy and collapse the stabilization device 450 against the catheter 432 for withdrawal of the delivery device 430 from the patient.

FIG. 7 illustrates select components of an alternate delivery system 500 including a first delivery device 530 having a catheter 532 and a stabilization device 550, which can be any stabilization device disclosed herein. In this embodiment, the catheter 532 is a hollow tube having an open end 534, which can be used as an introducer to guide and position a second delivery device 570 of the system 500 supporting an apparatus 540 that is to be positioned at a target site (e.g., left atrium). In this way, the stabilization device 550 supports not only the catheter 532 but the second delivery device 570. The second delivery device 570 is schematically illustrated and can be any of the kind capable of transcatheter delivery of a prosthesis, ablation or other apparatus. Examples of suitable delivery devices are disclosed in U.S. Pat. No. 8,641,704 (Werneth et al.), the disclosure of which is herein incorporated by reference in its entirety.

It is to be understood that any of the disclosed stabilization devices can be used with any of the disclosed delivery devices. In other words, the type of stabilization device used is not dependent on the type of delivery system, device or apparatus secured to a delivery device.

Although the present disclosure has been described with reference to preferred embodiments, workers skilled in the art will recognize that changes can be made in form and detail without departing from the spirit and scope of the present disclosure.

What is claimed is:

1. A delivery system for transseptal delivery of an apparatus to a human heart from a vena cava of the human heart to a right atrium, through a septal wall and into a left atrium; the delivery system comprising:
a first delivery device including a catheter and a stabilization device affixed thereto at a position on the catheter such that the stabilization device can be deployed within the vena cava when a distal end of the catheter is positioned within the left atrium; the stabilization device having a delivery position and a deployed position; wherein a diameter of the stabilization device is greater in the deployed position than in the delivery position; wherein the stabilization device is configured to define at least one blood flow path through the stabilization device when in the deployed position.

2. The delivery system of claim 1, wherein the catheter is hollow and has an open distal end though which a second delivery device can be delivered to the left atrium.

3. The delivery system of claim 1, wherein the catheter is configured to retain an apparatus at the distal end of the catheter.

4. The delivery system of claim 3, wherein the apparatus is a stented prosthetic heart valve.

5. The delivery system of claim 3, wherein the apparatus is an ablation apparatus.

6. The delivery system of claim 1, wherein the catheter is at least partially covered with a sheath.

7. The delivery system of claim 1, wherein the stabilization device has a generally triangular cross section when in the deployed position.

8. The delivery system of claim 1, wherein the stabilization device is inflatable to transition from the delivery position to the deployed position.

9. The delivery system of claim 1, wherein the stabilization device includes a plurality of ribs that bow outwardly with respect to the catheter when in the deployed position.

10. The delivery system of claim 1, wherein the stabilization device is collapsed against the catheter when in the delivery position.

11. The delivery system of claim 1, wherein in the deployed position, the stabilization device includes a generally cylindrical body and a plurality of webs extending between the cylindrical body and the catheter.

12. The delivery system of claim 11, wherein the stabilization device includes three webs.

13. A method of stabilizing a catheter during transseptal access of a left atrium of a heart of a patient; the method comprising the steps of:
providing a first delivery device including:
a catheter and a stabilization device affixed thereto; the stabilization device having a delivery position and a deployed position; wherein a diameter of the stabilization device is greater in the deployed position than in the delivery position;
with the stabilization device in the delivery position, inserting the first delivery device into a vascular system of the patient to position the stabilization device proximate a vena cava portion of the vascular system; and
actuating the stabilization device such that the diameter of the stabilization expands into a deployed position to engage the vena cava.

14. The method of claim 13, wherein the catheter is a hollow tube; the method further comprising the step of inserting a second delivery device supporting an apparatus through the catheter such that the apparatus is positioned within the left atrium.

15. The method of claim 14, wherein the apparatus is a stented prosthetic heart valve.

16. The method of claim 14, wherein the apparatus is an ablation apparatus.

17. The method of claim 13, wherein the stabilization device defines a plurality of blood flow passages when in the deployed position.

18. The method of claim 17, wherein the blood flow passages are formed within the stabilization device when the stabilization device is deployed.

19. The method of claim 13, wherein the stabilization device has a generally triangular cross-section in the deployed position.

20. The method of claim 13, comprising the step of inflating the stabilization device to transition the stabilization device from the delivery position to the deployed position.

21. The method of claim 13, wherein the stabilization device includes a plurality of ribs; wherein the method includes the step of causing the ribs to bow outwardly with respect to the catheter to transition the stabilization device from the delivery position to the deployed position.

22. The method of claim 13, wherein the stabilization device includes a cylindrical body and a plurality of webs extending between the cylindrical body and the catheter.

23. The method of claim 13, further comprising the step of transitioning the stabilization device from the deployed position to the delivery position and withdrawing the first delivery device from the patient.
